(19) United States
(12) Patent Application Publication
(10) Pub. No.: US 2011/0137155 A1
(43) Pub. Date: Jun. 9, 2011

(54) DELIVERY DEVICE FOR LOCALIZED DELIVERY OF A THERAPEUTIC AGENT

(75) Inventors: Jan WEBER, Maastricht (NL); Kent HARRISON, Maple Grove, MN (US); Aiden FLANAGAN, Kilcolgan (IE)

(73) Assignee: BOSTON SCIENTIFIC SCIMED, INC., Maple Grove, MN (US)

(21) Appl. No.: 12/962,962

(22) Filed: Dec. 8, 2010

Related U.S. Application Data
(60) Provisional application No. 61/267,944, filed on Dec. 9, 2009.

(51) Int. Cl. A61M 25/10; A61B 5/05

(52) U.S. Cl. 600/424; 604/65; 604/509; 604/103.02

(57) ABSTRACT

Therapeutic agent delivery devices and methods for delivering a therapeutic agent to a target location as well as methods for determining the location of a lesion on a vessel wall are disclosed. Various embodiments disclose an expandable member comprising a drug delivery matrix for selectively delivering a therapeutic agent to a lesion on a vessel wall. The drug delivery matrix may comprise one or more sensors and an electroactive polymer for releasing the therapeutic agent. Other embodiments disclose an expandable member comprising a plurality of radially-expanding flexible walls forming a plurality of channels for selectively delivering therapeutic agent to a target area adjacent one or more of the channels. Detecting a lesion may comprise using a plurality of Hall effect sensors disposed on a distal end of a catheter.

### CROSS REFERENCE TO RELATED APPLICATION

The present application claims priority to U.S. provisional application Ser. No. 61/267,944 filed Dec. 9, 2009, the disclosure of which is incorporated herein by reference in its entirety.

### TECHNICAL FIELD

The present disclosure relates to the delivery of a therapeutic agent, for example to the interior walls of a vessel such as a blood vessel, via a therapeutic agent delivery device, and to detection of lesions on the walls.

### BACKGROUND INFORMATION

The deployment in the body of medications and other substances, such as materials useful in tracking biological processes through non-invasive imaging techniques, is an oft repeated and advantageous procedure performed during the practice of modern medicine. Such substances may be deployed through non-invasive procedures such as endoscopy and vascular catheterization, as well as through more invasive procedures that require larger incisions into the body of a patient.

In conventional minimally-invasive medical treatment, medical instruments are steered by physicians to the location within the patient's body at which the procedure is to be performed, using, for example, images from optical devices located at the end of the instruments' lumens or from non-invasive imaging techniques. Once placed at the desired site, the device at the distal end of the instrument can be actuated by the physician to perform the procedure.

These procedures often require careful, time-consuming monitoring of the placement of the instrument tip within the body. Even with such care, however, limitations on the quality of the available images and obstruction of views by surrounding tissues or fluids can degrade the accuracy of placement of the instrument. Such difficulties can result in less than optimal injection, infusion, inflation or sample collection. Moreover, even if positioned properly, the instrument might be aligned with areas in which performance of the medical procedure would not be desired, such as where an asymmetric plaque deposit inside a blood vessel would render infusion delivery or angioplasty ineffective or potentially dangerous.

U.S. Patent Application Publication No. 2004/0102733 to Naimark et al., which is expressly incorporated herein by reference, presents a solution to some of these inefficiencies. That publication describes a minimally-invasive smart device which can detect environmental conditions in the vicinity of a target site within a patient's body and determine whether the medical device on the distal end of the instrument should be activated to perform, or be inhibited from performing, a desired minimally-invasive medical procedure.

Despite these advances, a need exists for more accurate detection of diseased locations and localized delivery of therapeutic agents as well as for better and more reliable overall structural design of therapeutic agent delivery systems and the mechanisms that support their functions.

### SUMMARY OF THE DISCLOSURE

The disclosure is directed to improvements in devices for delivery of a therapeutic agent to a target location, such as the inside of a vessel, as well as in devices for detection of lesions, such as on the inside of a vessel.

In one embodiment of the disclosure, a therapeutic agent delivery device is provided comprising an elongate member having a distal end, an expandable member disposed on the distal end of the elongate member and a drug delivery matrix disposed on at least a portion of the expandable member. The drug delivery matrix comprises one or more drug delivery areas with each drug delivery area comprising an electroactive polymer, one or more sensors adapted to detect a condition of a target location on a vessel wall, and one or more conductive elements for transmitting one or more signals from the one or more sensors and for transmitting one or more signals to the electroactive polymer of the one or more drug delivery areas. In this embodiment, when a first sensor of the one or more sensors detects the condition of the target location, the first sensor transmits one or more signals, and based on such detection, one or more signals are transmitted to one or more drug delivery areas of the one or more drug delivery areas, thereby causing the therapeutic agent to be delivered from the electroactive polymer of the one or more drug delivery areas to the target location.

A disclosed further embodiment provides a method of delivering a therapeutic agent to a target location, the method comprising providing a therapeutic agent delivery device comprising an elongate member having a distal end, an expandable member disposed on the distal end of the elongate member and a drug delivery matrix disposed on at least a portion of the expandable member. The drug delivery matrix comprises one or more drug delivery areas with each drug delivery area comprising an electroactive polymer, one or more sensors adapted to detect a condition of the target location on a vessel wall, and one or more conductive elements for transmitting one or more signals from the one or more sensors and for transmitting one or more signals to the electroactive polymer of the one or more drug delivery areas. The method further comprises positioning the device in the vessel, detecting the condition of the target location and transmitting one or more signals from a first sensor of the one or more sensors that detected the condition, and, based on the detection, transmitting one or more signals to one or more drug delivery areas, thereby causing the therapeutic agent to be delivered from the electroactive polymer of the one or more drug delivery areas to the target location.

A disclosed further embodiment provides a method of delivering a therapeutic agent to a target location, the method comprising determining one or more target drug delivery areas on a vessel wall and providing a therapeutic agent delivery device comprising an elongate member having a distal end, an expandable member disposed on the distal end of the elongate member and a drug delivery matrix disposed on at least a portion of the expandable member. The drug delivery matrix comprises one or more drug delivery areas, with each drug delivery area comprising an electroactive polymer, and one or more conductive elements for transmitting a signal to the electroactive polymer of the one or more drug delivery areas. The method further comprises positioning the therapeutic agent delivery device in the vessel and transmitting a signal to one or more drug delivery areas, thereby causing the therapeutic agent to be delivered from the electroactive polymer of the one or more drug delivery areas to the target location.

A disclosed further embodiment provides a therapeutic agent delivery device comprising an elongate member having a distal end and an expandable member disposed on the distal end of the elongate member. The expandable member comprises a plurality of adjacent radially-expanding flexible walls that extend longitudinally in an axial direction along the length of the expandable member, the flexible walls forming a plurality of channels. The device further comprises a delivery lumen for delivering therapeutic agent to one or more of the plurality of channels. In this embodiment, the therapeutic agent is delivered from the delivery lumen to at least a first channel selected from the plurality of channels to a target location.

A disclosed further embodiment provides a method of delivering a therapeutic agent to a target location, the method comprising providing a therapeutic agent delivery device comprising an elongate member having a distal end, an expandable member disposed on the distal end of the elongate member, the expandable member comprising a plurality of adjacent radially-expanding flexible walls that extend longitudinally in an axial direction along the length of the expandable member, the flexible walls forming a plurality of channels, and a delivery lumen for delivering therapeutic agent. The method further comprises delivering the therapeutic agent from the delivery lumen to a first channel of the plurality of channels to a target location.

A disclosed further embodiment provides a method of determining the location of a lesion on a vessel wall, the method comprising flushing the vessel with a detectable agent and providing a lesion detection device comprising an elongate member having a distal end and a plurality of sensors disposed on the distal end of the elongate member, the plurality of sensors adapted to sense the detectable agent. The method further comprises positioning the lesion detection device in the vessel and determining a location of the lesion on the vessel wall based on signals received by the plurality of sensors from the detectable agent.

Depending on the embodiment, a device and/or method as disclosed herein can have advantages including reduced loss of therapeutic agent during and/or after the procedure, reduced delivery and/or application of therapeutic agent at undesired times or to undesired locations, simplicity of design, reduced procedural complications, improved ease of use, and/or improved overall performance during and/or after the procedure. These and other features and advantages of the disclosed devices and methods are described in, or apparent from, the following detailed description of various exemplary embodiments.

### BRIEF DESCRIPTION OF THE DRAWINGS

Various embodiments will be more readily understood through the following detailed description, with reference to the accompanying drawings, in which:

FIG. 1 is a schematic view of a therapeutic agent delivery device according to an embodiment of the present disclosure.

FIG. 2 is a schematic view of a drug delivery matrix of the therapeutic agent delivery device illustrated in FIG. 1.

FIG. 3 is a perspective view of one side of a strip that can be used in a therapeutic agent delivery device according to an embodiment of the present disclosure.

FIG. 4 is a perspective view of the other side of the strip of FIG. 3.

FIG. 5 is a perspective view of a therapeutic agent delivery device incorporating the strip of FIGS. 3 and 4.

FIG. 6 is a cross-sectional view taken along the line 6-6 in FIG. 5.

FIG. 7 is a perspective view of a therapeutic agent delivery device according to another embodiment of the present disclosure.

FIG. 8 is a cross-sectional view taken along the line 8-8 in FIG. 7.

FIG. 9 is a longitudinal cross-sectional view of an inner tube of FIG. 7.

FIG. 10 is an end view of the inner tube of FIG. 7.

FIG. 11 is a schematic view of a therapeutic agent delivery device according to another embodiment of the present disclosure.

FIG. 12 is a schematic cross-sectional view of the therapeutic agent delivery device illustrated in FIG. 11 in a retracted position.

FIG. 13 is a perspective view of box “A” of the therapeutic agent delivery device illustrated in FIG. 11.

FIG. 14 is a perspective view of a therapeutic agent delivery device according to another embodiment of the present disclosure.

FIG. 15 is a cross-sectional view taken along the line 15-15 in FIG. 14.

FIG. 16 is a schematic view of a lesion detection device in accordance with one embodiment of the present disclosure.

### DETAILED DESCRIPTION

For a general understanding of the features of the illustrated embodiments of the disclosure, reference is made to the drawings. In the drawings, like reference numerals have been used throughout to designate like elements.

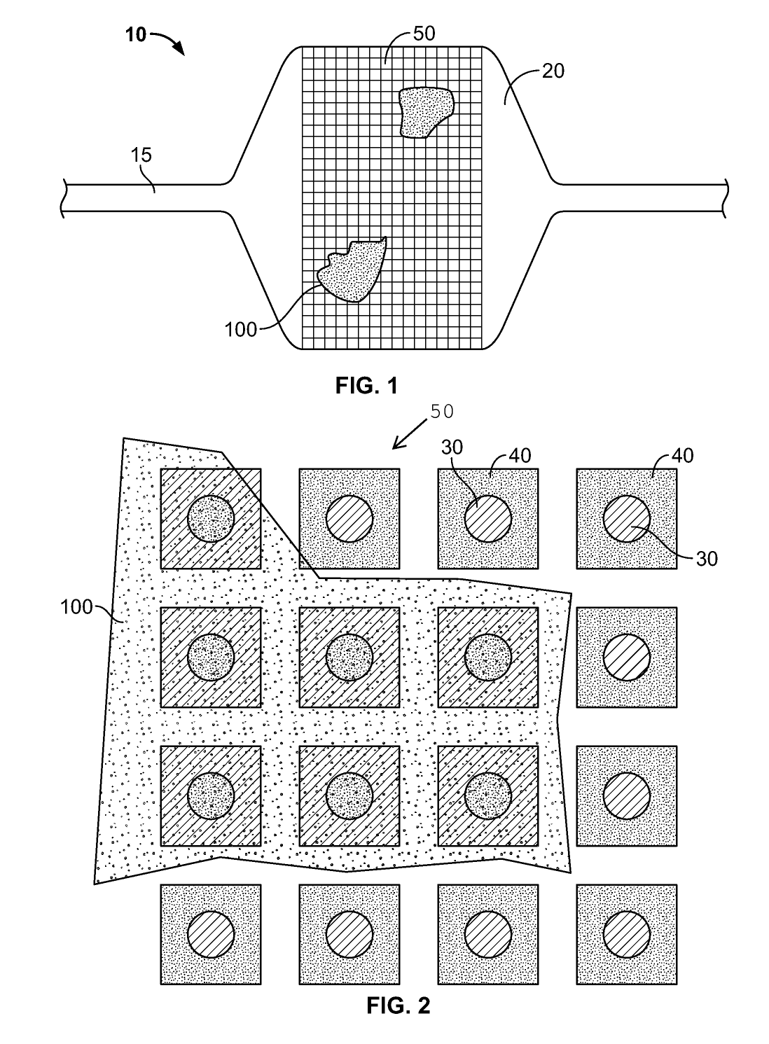

As illustrated in FIG. 1, a therapeutic agent delivery device 10 according to a first embodiment includes an elongate member in the form of a catheter 15 having a distal end and an expandable member 20. Drug delivery matrix 50 is disposed on at least a portion of an outer surface of the expandable member 20. The expandable member 20 may be mounted on the distal end of a catheter 15 for delivery to a desired target location 100 such as, for example, to the vasculature of the human body.

As illustrated in FIG. 2, the drug delivery matrix 50 includes a plurality of drug delivery areas 40. In this embodiment, each drug delivery area 40 comprises an electroactive polymer. The drug delivery matrix 50 also includes a plurality of sensors 30 adapted to detect a condition of the target location 100 on a vessel wall. In this embodiment, the drug delivery matrix 50 also includes a plurality of conductive elements (not shown) for transmitting one or more signals from the sensors 30 and for transmitting one or more signals to the electroactive polymer of the plurality of drug delivery areas 40. In certain embodiments, the signals are electrical signals. However, other signals such as, for example, radio signals may be used.

In the embodiment of FIG. 2, release of therapeutic agent from the electroactive polymer is triggered by an electronic signal. The strength of the signal is not particularly limited. In certain embodiments, the electrical signal is 1 Volt, micro Ampere. However, other suitable signals are within the scope and spirit of this disclosure.

In the embodiment of FIG. 2, the sensors 30 generally correspond to drug delivery areas 40. As illustrated in FIG. 2, the sensors 30 and drug delivery areas 40 correspond in a one-to-one relationship. However, multiple sensors 30 may correlate to a single drug delivery area 40, and multiple drug delivery areas 40 may correlate to a single sensor 30. Moreover, it is contemplated that some drug delivery areas 40 may not have corresponding sensors and may rely solely on communication with other drug delivery areas 40 for triggering, as disclosed herein.

The condition of the target location that is sensed by the sensor 30 can be any medical condition relevant to the disease to be treated. For purposes of this disclosure, the condition will be described with respect to plaque or a lesion on the interior wall of a blood vessel commensurate with a cardiovascular condition. Other conditions such as, for example, ulcers or tumors can be detected with sensors within the scope and spirit of this disclosure.

In embodiments such as those illustrated in FIGS. 1 and 2, when a condition to be sensed is present in the vessel adjacent a first sensor 30, the first sensor 30 detects the condition. Once the condition has been detected, the first sensor 30 transmits a signal. The signal may be directly transmitted to one or more drug delivery areas of the plurality of drug delivery areas 40, or the signal may be transmitted to another device or processor by which one or more signals is in turn transmitted to one or more drug delivery areas of the plurality of drug delivery areas 40. When a signal is transmitted to a drug delivery area 40, it thereby causes the therapeutic agent to be delivered from the electroactive polymer of the drug delivery area 40 to the adjacent target location 100. By way of analogy, for purposes of example only, the drug delivery areas 40 may act as drug releasing islands containing an electroactive polymer. In this manner, several individual islands are formed across the balloon surface as described in U.S. Provisional Patent Application 61/074,456, which is expressly incorporated herein by reference.

In another embodiment, the one or more drug delivery areas 40 are also adapted to communicate with one or more other drug delivery areas of the plurality of drug delivery areas 40. In this embodiment, when a signal is transmitted to the one or more drug delivery areas 40, these drug delivery areas may communicate the signal to one or more other drug delivery areas 40, thereby causing the therapeutic agent to release from an electroactive polymer of the one or more other drug delivery areas 40 and be delivered to the target location 100. In this manner, the drug delivery matrix 50 is able to efficiently adapt to various sizes and shapes of target lesions 100 and deliver therapeutic agent to “fringe” areas of the matrix where the condition may be too weak to trigger the sensor 30 but where it would be advantageous to still supply drug.

The types of sensors used are not particularly limited. Micro-sized and nano-sized sensors suitable for use in biological applications are well known in the art. In certain embodiments, the sensors may comprise at least one of mechanical, environmental and biochemical sensors. For example, the sensor may be a temperature sensor that measures the plaque temperature of a lesion. Plaque temperature has been shown to be correlated directly with inflammatory cell density. See Mohammad Madjid, MD, Morteza Naghavi, MD, Basit A. Malik, MD; Thermal Detection of Vulnerable Plaque; The American Journal of Cardiology, Volume 90, Issue 10, Supplement 3, 21 Nov. 2002, pages L36-L39. Another example is the use of pH value as a triggering parameter. It has been shown that unstable vulnerable plaques have a lower pH value than surrounding tissue. Miniature-sized pH sensors are also known in the art. See Olga Korostynska , Khalil Arshak, Edric Gill and Arousian Arshak; Review on State-of-the-art in Polymer Based pH Sensors; Sensors 2007, 7, 3027-3042. Other suitable sensors are within the scope and spirit of this disclosure.

In the embodiment illustrated in FIG. 1, the expandable member 20 may be a balloon. Any suitable material may be used for the balloon 20, such as, for example, a polymeric material. Angioplasty balloon materials have been the subject of a number of patents and patent applications including U.S. Patent Application Publication No. 2007/0208365 to Lee et al. and U.S. Patent Application Publication No. 2007/0208405 to Goodin et al. The disclosures of these applications are expressly incorporated herein by reference. The balloon 20 may be formed, for example, from a high durometer PEBAX®, such as PEBAX® 7233, 7033 or 6333 or NYLON 12®.

Examples of other polymeric materials from which the balloon 20 may be formed include polyethylene, HYTREL®, polyester, polyurethane, ABS (acrylonitrile-butadiene-styrene) block copolymer, ABS/Nylon blends, ABS/polycarbonate blends and combinations thereof, styrene-acrylonitrile block copolymers, other acrylonitrile copolymers, polyacrylamide, polyacrylates, polyacrylsulfones polyester/polycaprolactone blends, polyetheretherketone (PEEK), polyethersulfone (PES), polyetherimide (PEI), polyetherketone (PEK), polymethylpentene, polyphenylene ether, polyphenylene sulfide, polyolefins such as polyethylene and polypropylene, olefin copolymers, such as ethylene-propylene copolymer, ethylene-vinyl acetate copolymers, ethylene-vinyl alcohol copolymers and polyolefin ionomers, polyvinyl chloride, polycaprolactam, N-vinyl-pyrrolidone, polyurethanes and polysiloxanes.

Electroactive polymers are members of a family of polymers referred to as “conducting polymers.” They are a class of polymers characterized by their ability to change shape in response to electrical stimulation. They expand and contract upon application of an appropriate electrical potential. They typically structurally feature a conjugated backbone and have the ability to increase electrical conductivity under oxidation or reduction. In an example embodiment, the electroactive polymer may be polypyrrole. Polypyrrole exhibits superior stability under physiological conditions. The structure of polypyrrole is depicted below:

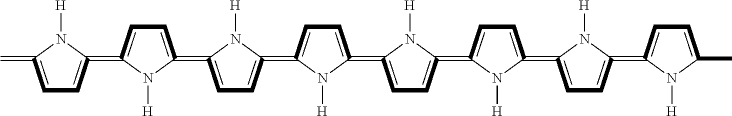

Known derivatives of polypyrrole include the following substituted polymers: poly(N-methylpyrrole), poly(N-butylpyrrole), poly[N-(2-cyanoethyl)pyrrole], poly[N-(2-carboxyethyl)pyrrole], poly(N-phenylpyrrole), poly[N(6-hydroxyhexyl)pyrrole] and poly[N-(6-tetrahydropyranylhexyl)pyrrole], among others. In addition to polypyrrole, other suitable conducting polymers, including analogs of polypyrrole, that exhibit suitable contractile or expansile properties may be used within the scope of the disclosure.

In one embodiment, the electroactive polymer is deposited, for example, by electro polymerization on an electrode. In such an embodiment, the polymer balloon surface may be covered with a patterned electrode using a sputtering process in combination with a mask. In another embodiment, the electroactive polymer can be deposited by an inkjet printing process.

The plurality of conductive elements may be configured in any suitable manner and may be around the outer surface of the expandable member 20. For example, the conductive elements may connect drug delivery areas 40 in a one-to-one relationship with adjacent drug delivery areas 40, or the conductive elements may be configured to connect with non-adjacent drug delivery areas 40 via a multiplexing scheme. In some embodiments, the conductive elements may comprise at least one of metal and polymer wiring. For example, the conductive elements may comprise Au, Ag, Pd, Pt, Fe, Mg or any suitable alloy thereof. Other suitable metals or metal alloys or conductive non-metal materials may be used for the conductive elements within the scope and spirit of this disclosure.

The therapeutic agent delivery device 10 according to these embodiments is practiced in the following manner with reference to FIGS. 1 and 2. An operator or physician, for example, inserts the delivery device 10 into a lumen of the human body by known techniques. For purposes of this disclosure, reference will be made to a vessel of the vasculature system. However, one of ordinary skill in the art will readily understand that the delivery device 10 may be used in another suitable lumen such as, for example, the human esophagus.

The operator or physician positions the delivery device 10 in the vessel by tracking the elongate member through the vessel until the expandable member 20 is at the desired position. Once in position, the delivery device 10 is activated. For example, the expandable member 20 may be expanded and the sensors 30 activated. Once activated, the sensors 30 of the plurality of drug delivery areas 40 detect any lesions 100 on the vessel wall. As illustrated in FIG. 2, the sensors 30 corresponding to the location of the lesion 100 detect the presence of the lesion by means disclosed herein. The lesion 100 may make direct contact with the sensors 30 or, alternatively, the sensors 30 may be configured to sense the presence of the lesion 100 without direct contact, as would be understood by one of ordinary skill in the art. At this point, the sensors 30 of the drug delivery areas 40 that detect the lesion 100 transmit one or more signals, either directly to the electroactive polymers of the corresponding drug delivery areas 40, or to another device or processor by which, in turn, one or more signals are sent to the electroactive polymers of the corresponding drug delivery areas 40. The one or more signals transmitted to the electroactive polymer of the drug delivery area 40 thereby activate the electroactive polymer, causing the therapeutic agent to be released from the electroactive polymer and to be delivered to the lesion or target location 100 for treatment. In this regard, each sensor 30 may act as an on-off signal such that once a drug delivery area 40 is activated, it will release the intended drug amount. In this way, therapeutic agent is delivered only to those target areas where therapeutic agent is desired, thereby avoiding delivery of therapeutic agent to other areas as well as avoiding waste of therapeutic agent. In certain embodiments, each drug delivery area 40 effected may also communicate a signal to one or more other drug delivery areas, thereby causing the therapeutic agent to be released from an electroactive polymer of that region or regions as well. In this way, the delivery of therapeutic agent can extend beyond the detected area, for example to a desired distance around the perimeter of the detected area.

In another embodiment, the target locations 100 on the vessel wall are detected or predetermined before the physician inserts the delivery device 10 into the vessel. In this embodiment, the three-dimensional location of the lesions on the vessel wall are mapped during a pre-scanning process using a scanning apparatus. The resulting data or map is then applied during use of the delivery device 10 by way of the delivery device 10 being coupled to or activated in accordance with the pre-scanned position and orientation data. The scanning apparatus may comprise any suitable device or devices known in the art of medical imaging. In embodiments, the pre-scan may be effectuated by X-Ray, CT, MRI or OCT scanning.

In certain embodiments, the pre-scan may be facilitated by first flushing the vessel with a detectable agent before scanning the vessel wall. In one example embodiment, the detectable agent is a super-paramagnetic iron particle. Super-paramagnetic iron particles have been coupled with polymer-lipid nanoparticles containing the antiangiogenic agent fumagillin and targeted against αvβ3 integrins of proliferating neovasculature in unstable plaques. For example, vascular cell adhesion molecule 1 (VCAM-1) is a known coupling agent. See Nahrendorf, M., Jaffer, F. A., Kelly, K. A., et al., Noninvasive Vascular Cell Adhesion Molecule-1 Imaging Identifies Inflammatory Activation of Cells in Atherosclerosis, Circulation 114:1504-1511 (2006). Noninvasive vascular cell adhesion molecule-1 imaging identifies inflammatory activation of cells in atherosclerosis. The detectable agent may also be a particle that assembles in macrophages, for example, that are present in inflamed atherosclerotic plaques. Several approaches to the use of such particles are known in the art. See Pavel Broz, Stephan Marsch and Patrick Hunzikel; Targeting of Vulnerable Plaque Macrophages with Polymer-Based Nanostructures; Trends in Cardiovascular Medicine, Volume 17, Issue 6, August 2007, pages 190-196.

Once in position, the delivery device 10 is activated to locally release the therapeutic agent to only those portions of the vessel that were predetermined to have lesions. The activation may be effected by suitable means. The drug delivery matrix 50 may be activated automatically similar to the embodiment of FIGS. 1 and 2, in which sensors 30 transmit a signal, and a signal is transmitted to the drug delivery areas 40. Alternatively, the delivery device 10 may be configured with microprocessors that store the pre-scanned data and automatically deliver a signal to the drug delivery matrix 40. The drug delivery matrix 50 may also be activated manually or by other suitable means.

In another embodiment, an imaging apparatus is provided that allows the physician to track the position of the delivery device 10 in the vessel. In this manner, the physician uses the pre-scanned data as an aid in aligning the delivery device 10 axially and rotationally. The physician may also manually send a signal to the drug delivery matrix 50 based on an external imaging apparatus once the delivery device 10 is in position. It is contemplated that positioning the delivery device 10 using the imaging apparatus will be facilitated by placing markers such as, for example, X-Ray or MRI markers, adjacent to or on the surface of the expandable member 20. Further, internal scanners, such as, for example, MRI imaging catheters using micro-coils or OCT, facilitate detailed imaging of the vessel wall. In this instance, the microcoil allows high resolution images of the vessel wall and as such enables detection of the SPIO particles after which the operator can activate the drug delivery areas on the balloon surface that are located opposed to the affected area.

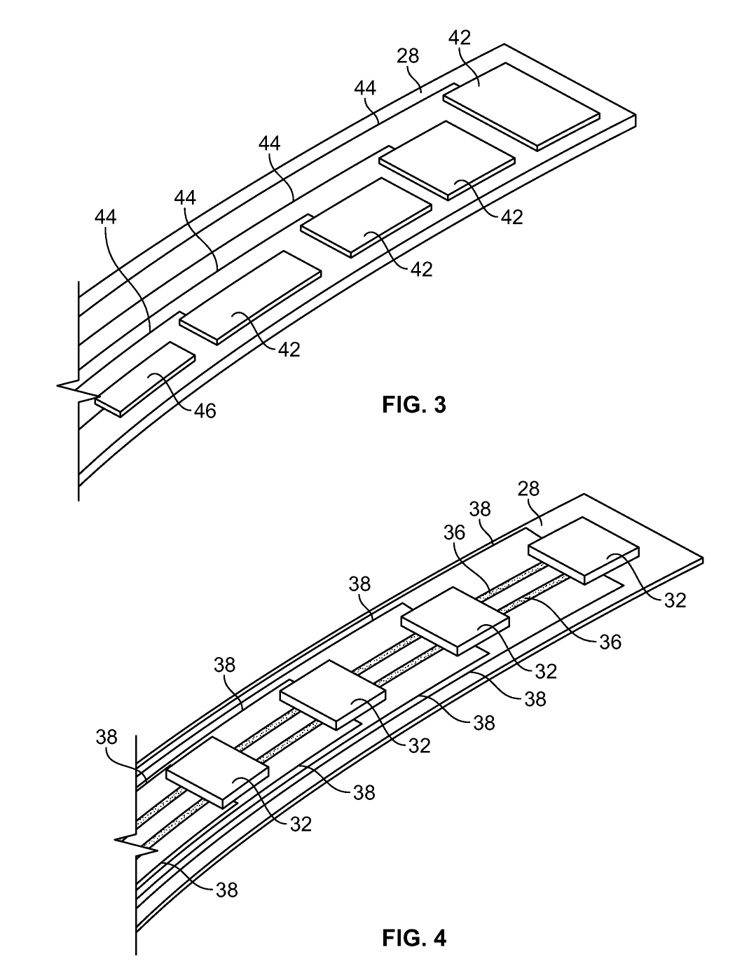

FIGS. 3 through 6 illustrate a therapeutic agent delivery device 12 according to another embodiment. In this embodiment, instead of directly mounting the sensors and electroactive polymer on the surface of the balloon or expandable member 22, the therapeutic agent delivery device 12 is made by first making a flexible strip 28 containing the sensors and electroactive polymer elements and mounting this strip 28 to the outside of the balloon or expandable member 22.

FIG. 3 shows one side of a strip 28 that can be used in such an embodiment, and FIG. 4 shows the other side of the strip 28. The strips 28 may be made, for example, of a suitable polymer material. For example, the strips may be made of nylon or VESTAMID® that is extruded and cut.

On one side of the strip 28, shown in FIG. 4, conductive elements in the form of conductive lines 36, 38 are placed or formed, for example, by printing using a suitable conductive material. The conductive lines 36, 38 may include, for example, two conductive lines 36 for power supply to the sensors 32 (positive and negative) and a plurality of conductive lines 38 for signal retrieval from the sensors 32. Sensors 32, such as micro Hall sensors as described herein, may be placed and bonded, e.g., glued, on the strip 28 as shown. A connection is made between the conductive lines 36, 38 and the sensors 32, which may be accomplished using a conductive epoxy.

On the opposite side of the strip 28, shown in FIG. 3, conductive elements in the form of conductive lines 44 are placed or formed, similar to the placement or formation of the conductive lines 36, 38 on the side of the strip shown in FIG. 4. In addition, a plurality of islands of conductive material, for example silver or another suitable material, may be placed or formed on this side of the strip for forming the drug delivery areas 42, with each conductive line 44 terminating in a conductive island for a drug delivery area 42. The spacing and placement of the islands for the drug delivery areas 42 generally correspond to that of the sensors 32 on the other side of the strip 28. A counter electrode 46 is also placed or formed on this side of the strip 28 to facilitate activation of the electroactive polymer.

The drug delivery areas 42 are formed, for example, of an electroactive polymer as described herein. As just one possible example for this embodiment, the electroactive polymer may be polypyrrole (PPy), and the therapeutic agent may be charged Dexamethsone (Dex), a synthetic anti-inflammatory drug. Dexamethasone disodium phosphate can be obtained from Sigma-Aldrich Co. Other suitable therapeutic agents and electroactive polymers may be used, including, for example, therapeutic agents and electroactive polymers as described in U.S. Provisional Patent Application 61/074,456, which, as mentioned above, is incorporated herein by reference. The drug delivery areas 42 may be formed, for example, by growing PPy/Dex film potentiostatically on the silver islands or by another suitable method.

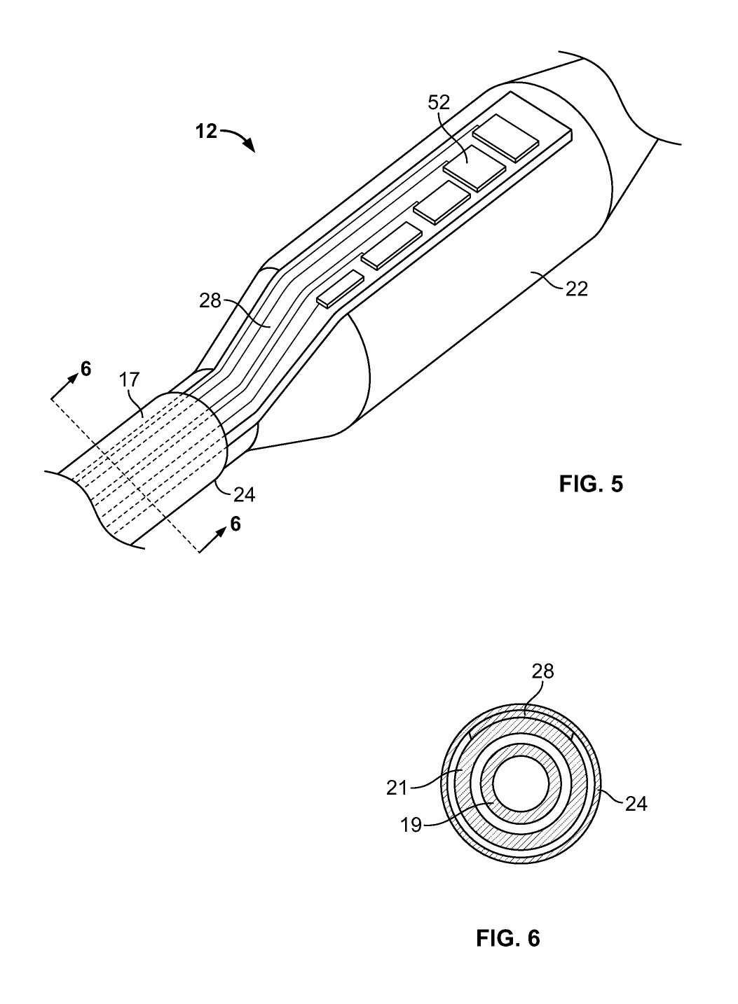

As shown in FIG. 5, once the strip 28 is formed with the sensors 32 and drug delivery areas 42 in place, the strip 28 may be attached, for example glued or otherwise bonded, to the balloon or expandable member 22 of the therapeutic agent delivery device 12. In this example embodiment, the strip 28 is glued onto the expandable member 22 with the sensors 32 facing the expandable member 22, such that the drug delivery areas 42 face outward.

The remainder of the strip 28 can run substantially along the length of the elongate member, which may be in the form of a catheter 17. The signals from the sensors 32 can be transmitted by conductive lines 38 to a device or processor outside of the body, thereby activating the transmission of signals by conductive lines 44 to activate the release of therapeutic agent by the electroactive polymer at the drug delivery areas 42.

While the distal end of the strip 28 is mounted on the expandable member 22, the portion of the strip 28 that runs along the length of the elongate member or catheter tubing may be mounted thereon using a heat shrink tube 24. As shown in cross-sectional view in FIG. 6, in this illustrated embodiment, the catheter has an inner tube 19 and an outer tube 21, and the strip 28 is held against the outer surface of the outer tube 21 by the heat shrink tube 24.

As shown in FIG. 5, once formed, the therapeutic agent delivery device 12 has a drug delivery matrix 52 disposed on at least a portion of an outer surface of the expandable member 22. The drug delivery matrix 52 comprises a plurality of drug delivery areas 42, with each drug delivery area 42 comprising an electroactive polymer, and a plurality of sensors 32 adapted to detect a condition of a target location on a vessel wall, as described herein. The therapeutic agent delivery device 12 also comprises conductive elements 38 for transmitting one or more signals from the sensors 32 and conductive elements 44 for transmitting one or more signals to the electroactive polymer of the plurality of drug delivery areas 42.

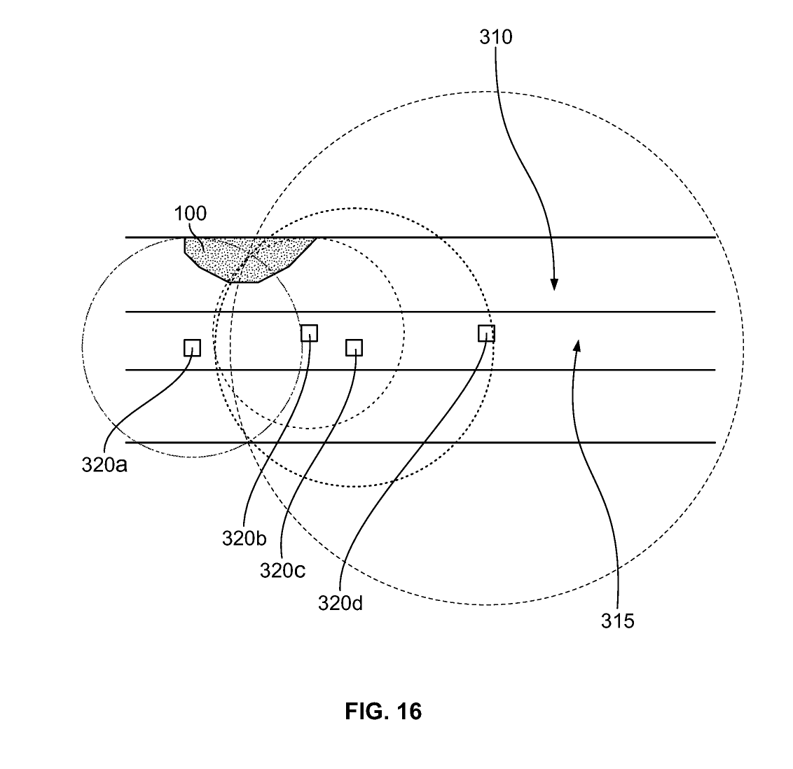

The therapeutic agent delivery device 12 is used in a similar manner as described herein with respect to FIGS. 1 and 2. An operator or physician, for example, inserts the delivery device 12 into a lumen, for example tracking the elongate member through a vessel to position the expandable member 22 adjacent to an area to be treated. The expandable member 22 may be expanded and the sensors 32 activated such that the sensors 32 detect target areas 100 on the vessel wall. The detection of the target areas 100 by Hall sensors may be similar to that described herein with reference to FIG. 16. The sensors 32 may alternatively detect temperature or another suitable indicator as described herein. When the sensors 32 detect the condition, they transmit one or more signals through the conductive lines 38 to another device or processor by which, in turn, one or more signals are sent to the electroactive polymer of the corresponding drug delivery areas 42. The one or more signals transmitted to the electroactive polymer of the drug delivery areas 42 thereby activate the electroactive polymer, causing the therapeutic agent to be released from the electroactive polymer and to be delivered to the target location 100 for treatment.

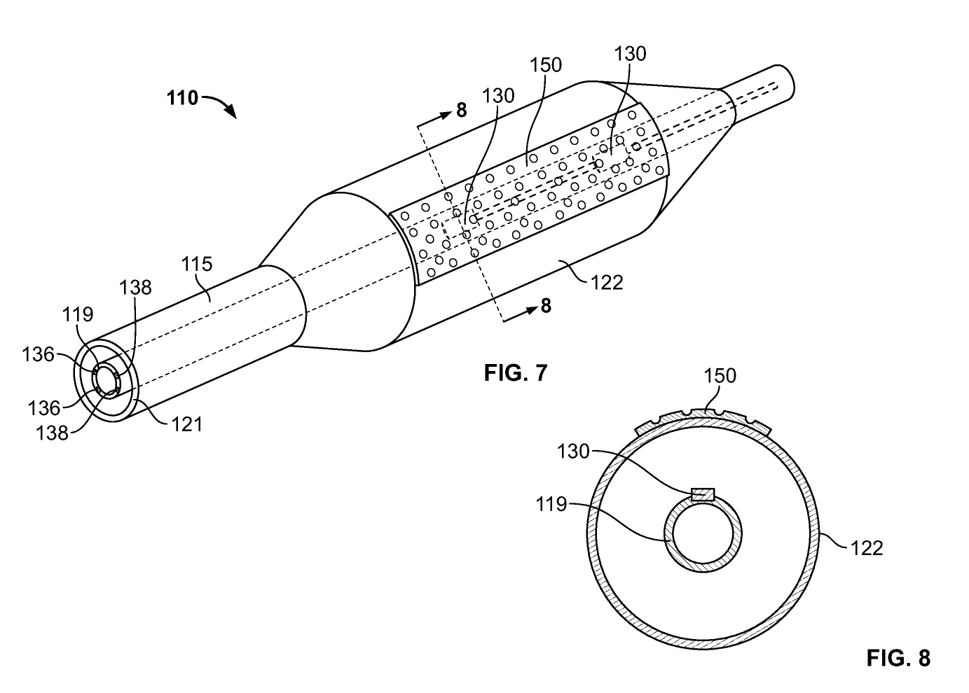

FIGS. 7 through 10 illustrate another embodiment of a therapeutic agent delivery device 110. The therapeutic agent delivery device 110 includes an elongate member in the form of a catheter 115 and a balloon or expandable member 122 mounted on the distal end of the catheter 115. The catheter 115 comprises an inner tube 119 and an outer tube 121.

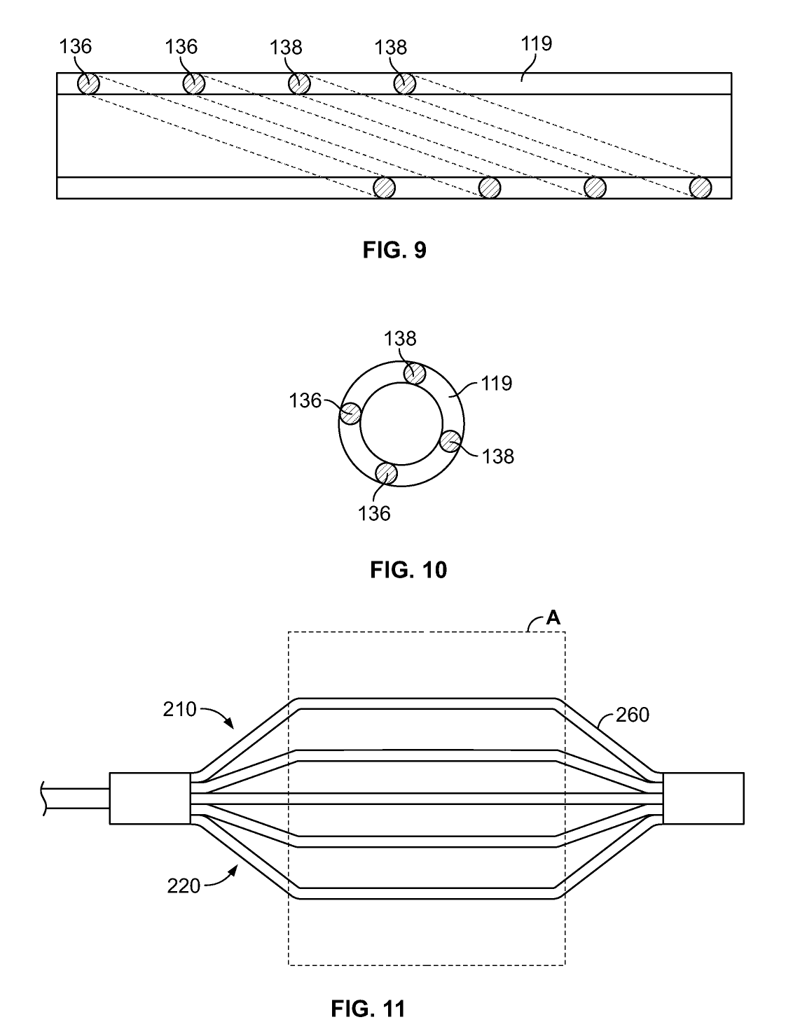

In this embodiment, the conductive elements for the sensors 130 are mounted in or on the inner tube 119. As can be seen in FIG. 9, which is a longitudinal cross-sectional view of the inner tube 119 of FIG. 7, as well as in FIG. 10, which is an end view of the inner tube 119 of FIG. 7, the conductive elements 136, 138 are illustrated as embedded within the wall of the inner tube 119. In this embodiment, the inner tube 119 is made with four conducting wires, for example of copper or another suitable conductor, inserted in the wall of the inner tube 119 to serve as the conductive elements 136, 138.

The sensors 130 can be micro Hall sensors or other sensors as described herein. The sensors 130 are mounted on the inner tube 119, for example in cavities that are formed, for example using an excimer laser, in the surface of the inner tube 119 to accommodate the sensors 130. In order to have a length of the conductive elements 136, 138 to attach to the sensors 130, the distal end of the inner tube 119 may be removed, for example using an excimer laser, by a process that removes the tubing but leaves the exposed wires. In this example, the conductive elements 136, 138 comprise two power supply conductive elements 136 and two conductive elements 138 for transmitting the signals from the sensors 130. In the illustrated embodiment comprising two sensors 130, both of the two sensors are attached to the power supply conductive elements 136 and each of the two sensors is attached to its own signal transmission conductive element 138. The exposed ends of the conductive elements 136, 138 are connected to the sensors 130, for example by soldering. The sensors 130 and conductive elements 136, 138 are folded backwards over the inner tube 119, and the sensors 130 are placed backside in the ablated cavities in the inner tube 119. A heat shrink tube may be shrunk over the sensors 130 and over the exposed conductive elements 136, 138. Also, a tip may be bonded to the distal end of the inner tube 119.

The inner tube 119 tube with the sensors 130 on it is positioned within the outer tube 121, with a distal portion of the inner tube 119 extending beyond the distal end of the outer tube 121. The balloon or expandable member 122 is attached, with the proximal end of the balloon or expandable member 122 affixed to the outer tube 121, and the distal end of the balloon or expandable member 122 affixed to the inner tube 119. A hub is affixed to the proximal part of the catheter 115.

The drug delivery matrix 150 can be a series of drug delivery areas positioned, for example, on one side of the balloon or expandable member 122. To place the drug delivery matrix 150 on the balloon or expandable member 122, the balloon or expandable member 122 is inflated or expanded, at which time the drug delivery matrix 150 is applied. The drug delivery matrix 150 may be applied to the same side of the device where the sensors 130 are positioned. The balloon or expandable member 122 is then deflated or brought back down to its unexpanded size for use.

During a procedure using the therapeutic agent delivery device 110, a patient may be infused intravenously with super-paramagnetic iron particles as described herein, and the patient may be scanned by MRI to locate the vulnerable plaques. A map is produced to be able to place the therapeutic agent delivery device 110 under fluoroscopy near the detected sites. Axial movement and rotation of the therapeutic agent delivery device 110 allows the physician to position the drug delivery matrix 150 based on the signals from the sensors 130. In this manner, the physician can superpose the drug delivery matrix 150 against the vulnerable plaque, after which the balloon is inflated to transfer the therapeutic agent to the target area. Thus, in this embodiment, only a part of the balloon carries a therapeutic agent, and the sensors allow the user to align the therapeutic agent to face the desired vessel wall section.

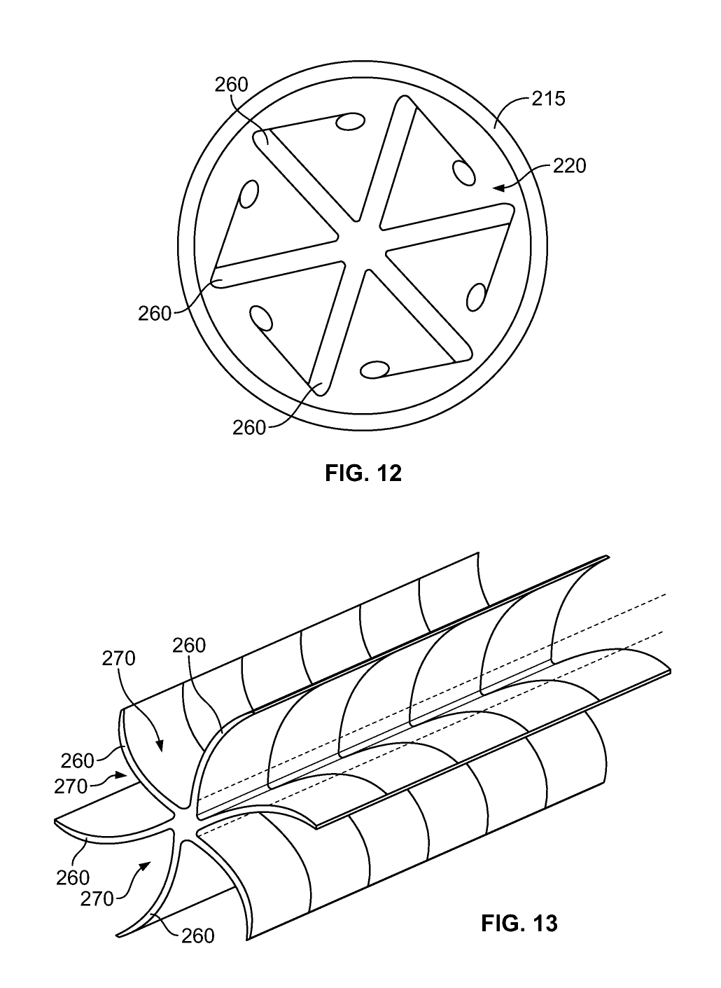

FIGS. 11-13 illustrate a therapeutic agent delivery device 210 according to another embodiment. The therapeutic agent delivery device 210 includes an elongate member in the form of a catheter having a distal end and an expandable member 220. The expandable member 220 is disposed on the distal end of the catheter. The expandable member 220 comprises a plurality of adjacent radially-expanding flexible walls 260 that extend longitudinally in an axial direction along the length of the expandable member 220. The flexible walls 260 form a plurality of channels 270, as best shown in FIG. 13. In this embodiment, the delivery device 210 also includes a delivery lumen (not shown) for selectively delivering the therapeutic agent to the plurality of channels. The channel 270 may be selected based on the location of the target lesion 100 on the vessel wall.

As shown in FIGS. 12 and 13, the expandable member 220 has a retracted position in an outer catheter 215 and an expanded position in a vessel. In practice, the expandable member 220 is moved in and out of the outer catheter 215 by actuating the outer catheter 215, or by actuating the elongate member or other structure to which the expandable member 220 is attached, proximally and distally in a longitudinal direction. In order to facilitate this movement, the plurality of adjacent radially-expanding flexible walls 260 may be tapered at a proximal end of the expandable member 220 to ease retraction of the expandable member 220 from the expanded position in the vessel to the retracted position in the outer catheter 215.

As illustrated in FIG. 12, the expandable member 220 has a retracted or collapsed position inside the outer catheter 215. When the expandable member 220 is deployed from the distal end of the outer catheter 215, it is expanded to its expanded position, as shown in FIG. 13. The expansion may be accomplished, for example, by self-expansion or expansion by inflation. In the expanded position, channels 270 are created in between adjacent radially-expanding flexible walls 260. In the embodiment of FIG. 13, in the expanded position the radially-expanding flexible walls 260 form a cross-sectional star-like shape, the distal-most radial ends of which may contact the walls of the vessel.

The flexible walls 260 may comprise a suitable flexible material, or a self-expanding or shape-memory material that is biocompatible. Non-limiting examples of flexible materials include, but are not limited to, stainless steels, such as 316, cobalt based alloys, such as MP35N or ELGILOY®, refractory metals, such as tantalum, and refractory metal alloys; precious metals, such as platinum or palladium, titanium alloys, such as high elasticity beta titanium, such as FLEXIUM®, nickel superalloys, and combinations thereof. Suitable shape-memory composite materials include Nitinol and others described in co-pending U.S. Patent Application Publication No. 2007/0200656 to Walak, which is expressly incorporated herein by reference.

The delivery device 210 may further comprise a plurality of sensors for locating the target location on a vessel wall. In such an embodiment, the sensors may be configured on the catheter or on a surface of the expandable member 220 according to one of the embodiments as described herein. In this regard, the sensors can be inserted inside the expandable member 220 to release drug from drug delivery areas on the surface of the expandable member (not shown).

In practice, the therapeutic agent delivery device 210 is positioned in a vessel at a target location. The physician moves the expandable member 220 into an expanded position, thus forming channels 270 in the vessel. One or more channels of the plurality of channels is then selected for drug delivery. Once the channel 270 is selected, the physician delivers the therapeutic agent from the delivery lumen through the first channel of the plurality of channels 270 to the target location 100. In this manner, therapeutic agent is delivered only within the confines of the selected channel 270 and not the entire vessel, as is often the case with conventional delivery devices. In this way, the device results in reduced loss of therapeutic agent and reduced delivery of therapeutic agent to undesired locations. In some embodiments, the channel 270 may be selected manually by a physician using an imaging apparatus, as disclosed herein. Alternatively, the channel 270 may be selected by using sensors to detect the location of a target lesion 100 on a vessel wall, as disclosed herein. In order to facilitate delivery to one or more specific channels 270, the physician may use a separate tube extending from outside of the patient to the desired channel(s). Additionally or alternatively, the outer catheter 215 may be sectioned into separate delivery lumens that correspond to the channels such that delivery through one or more lumens of the catheter results in delivery into one or more channels 270.

The delivery device 210 may incorporate the imaging and scanning features disclosed with respect to other embodiments described herein. In this regard, the delivery device 210 may be used with externally placed magnets to determine the location of the catheter within the body. For example the movement of the catheter due to the heart beat, breathing, and other body motions could be compensated for during imaging to provide still pictures such that if the catheter moves a distance x along the X-axis, then the image on the screen is moved by −xS, where S is a scaling factor, in order to compensate. Likewise, these features may be used to determine whether the delivery device 210 is in the correct position or to aid in its positioning to the desired site within the body.

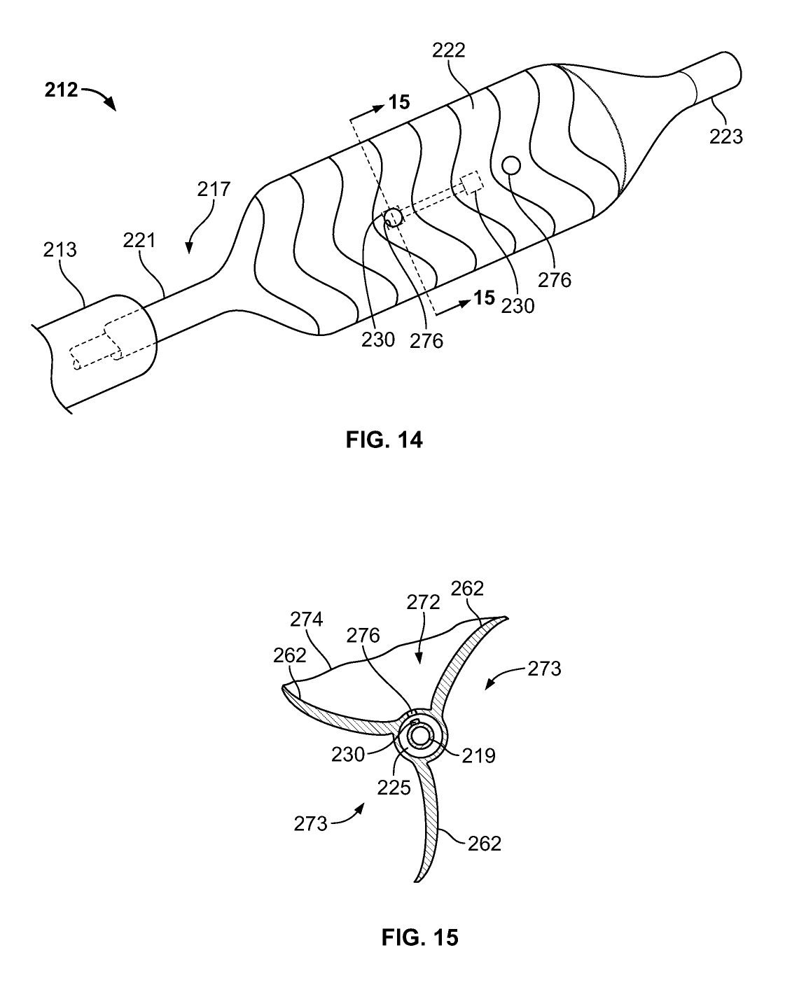

FIGS. 14 and 15 show a therapeutic agent delivery device 212 according to another embodiment. The therapeutic agent delivery device 212 comprises an elongate member in the form of a catheter 217 with an expandable member 222 mounted on the distal end of the catheter 217. Similar to the embodiment shown in FIGS. 11-13, the expandable member 222 comprises a plurality of adjacent radially-expanding flexible walls 262 that extend longitudinally in an axial direction along the length of the expandable member 222. The flexible walls 262 form a plurality of channels 272, 273, as best shown in FIG. 15. The expandable member 222 has a retracted position in a guide catheter 213 and an expanded position in a vessel. In a similar manner as described with respect to FIGS. 11-13, the expandable member 222 is moved into and out of the catheter 213 by either retracting the catheter 213 relative to the expandable member 222 or by pushing the expandable member 222 out of the distal end of the catheter 213. To ease retraction of the expandable member 222 from the expanded position in the vessel back into the retracted position in the catheter 213, the plurality of adjacent radially-expanding flexible walls 262 may be tapered at a proximal end of the expandable member 222.

When the expandable member 222 is deployed from the distal end of the catheter 213, it is expanded to its expanded position. The expansion may be accomplished by suitable means. For example, in the embodiment illustrated in FIGS. 14 and 15, the expansion is by self-expansion such that the expandable member 222 opens to its expanded configuration once released from the constraint of the catheter 213. In the expanded position, channels 272, 273 are created in between adjacent radially-expanding flexible walls 262. In the embodiment of FIGS. 14 and 15, in the expanded position, the distal-most radial ends of the radially-expanding flexible walls 260 contact the walls of the vessel.

In the embodiment shown in FIGS. 14 and 15, the delivery device 212 comprises a plurality of sensors 230 for locating the target location on a vessel wall. In this illustrated embodiment, the sensors 230 are configured on the inner tube 219 of the catheter 217. The sensors 230 may be mounted on the inner tube 219 of the catheter and coupled to conductive elements in a manner similar to that described herein with respect to FIGS. 7-10.

The catheter 217 further comprises an outer tube 221. The expandable member 222 is mounted on the distal end of the outer tube 221. The inner tube 219 extends through the outer tube 221 as well as through the,expandable member 222. The inner tube 219 and the expandable member 222 are joined together at their distal ends, at the tip 223 of the therapeutic agent delivery device 212.

The outer surface of the inner tube 219 is spaced from the inner surface of the outer tube 217 to leave a therapeutic agent delivery lumen 225. The therapeutic agent delivery lumen 225 extends from the proximal end of the therapeutic agent delivery device 212 to the expandable member 222, where it terminates at one or more ports 276.

In the illustrated embodiment, the ports 276 open into the channel 272, but no ports open into the other two channels 273. The channel 272 is closed off at its distal end by a membrane 274 extending between the adjacent radially-expanding flexible walls 262 on either side of the channel 272.

In practice, the therapeutic agent delivery device 212 is positioned in a vessel at a target location. Using the sensors in a similar manner to that described herein, for example with respect to the embodiment of FIGS. 7 through 10, the target site is detected. The therapeutic agent delivery device 212 is then oriented such that the channel 272 will be adjacent the target site once the expandable member 222 is deployed. Once the therapeutic agent delivery device 212 is oriented, the operator or physician moves the expandable member 222 into an expanded position, thus forming channels 272, 273. The channels 273 allow blood to continue to flow through the vessel. The therapeutic agent is then delivered from the proximal end of the catheter 217 through the therapeutic agent delivery lumen 225 and out of the ports 276 to the channel 272. In this manner, therapeutic agent is delivered substantially within the confines of the selected channel 272 and not throughout the entire vessel, as is often the case with conventional delivery devices. In this way, similar to other embodiments described herein, the device results in reduced loss of therapeutic agent and reduced delivery of therapeutic agent to undesired locations.

In alternative embodiments, the channel 272 may be oriented manually by a physician using an imaging apparatus, as disclosed herein. In such embodiments, the sensors 230 may be omitted.

Yet another embodiment is illustrated in FIG. 16. FIG. 16 illustrates a lesion detection device 310. The detection device 310 comprises an elongate member in the form of a catheter 315 having a distal end. In this embodiment, a plurality of sensors 320a, 320b, 320c and 320d are disposed on the distal end of the catheter. While four sensors are illustrated in FIG. 16, it readily will be understood that any suitable number of sensors may be used. In embodiments using trilateration as described herein, at least three sensors are used. The plurality of sensors 320 is adapted to sense a detectable agent. The detectable agent may be any suitable magnetic particle, as disclosed herein.

In this embodiment, the sensors 320 are Hall effect sensors. Hall effect sensors are capable of integration into microsystems. See Javad Frounchi, Michel Demierre, Zoran Randjelovic, Rade S. Popovic; ISSCC 2001/Integrated Hall Sensor Array Microsystem, Session 16/Integrated Mems and Display Drivers/16.3. Further, nano-sized (50 nm by 50 nm) Hall sensors are known in the art. See Adarsh Sandhu, Kouichi Kurosawai; 50 nm Hall Sensors for Room Temperature Scanning Hall Probe Microscopy; Japanese Journal of Applied Physics; Vol. 43, No. 2, 2004, pp. 777-778.

The Hall effect sensors may be arranged in a specific configuration in order to detect changes in magnetic field. In this manner, the sensors 320 identify the areas where the magnetic nanoparticles are accumulating. By flushing the vessel with magnetic nanoparticles as described herein, these particles accumulate in areas where the lesions are located in the vessel. The Hall effect sensor outputs a voltage or electrical signal in response to an applied magnetic field. The sensor is also directional in that it produces a stronger signal for incident magnetic field lines in one direction than for those at a different angle.

As illustrated in FIG. 16, sensors 320 are placed on the distal end of catheter 315 in the vicinity of a lesion infused with magnetic particles. Each sensor outputs a signal proportional to its distance to the lesion. Using trilateration, the position of the lesion can be pinpointed by mapping the intensity of the signals received. With reference to FIG. 16, the circles only intersect at one point corresponding to the lesion. By using error estimation and/or moving the sensors 320, the lesion size can be estimated. By using more sensors and varying their positions in three dimensions, multiple lesion can be pinpointed accurately as the catheter is moved along the vessel.

It is contemplated that the lesion detection device 310 may be combined with the therapeutic agent delivery devices of previous embodiments. For example, in certain embodiments, the sensors 320 may be disposed on the catheter of the embodiment illustrated in FIGS. 11-13. In this manner, the Hall effect sensors detect a lesion on a vessel wall, thus permitting selection of the appropriate channel 270 for delivery of therapeutic agent via automatic or manual means, as disclosed herein.

The following are some specific examples of devices that may be constructed in accordance with embodiments disclosed herein.

### EXAMPLE 1

A device as illustrated in FIGS. 3-6 can be made by mounting Hall sensors and electroactive polymer on a strip, and mounting the strip onto a balloon surface. First, a flexible polymer strip is made. Nylon strips (VESTAMID®) can be extruded and cut having dimensions 1 meter long (approximately the length of the catheter) by 2 mm wide and a thickness of 20 micrometers. The strips are cleaned with HNO3 for 10 minutes and rinsed with deionized water. On one side, 10 parallel conductive lines (100 micrometers wide and 2 micrometers high with a spacing of 50 micrometers) are printed using an aqueous silver nanoparticle dispension SP100 (PChem Associates Inc., Bensalem, Pa.) and a MD-K-130 printing system from Microdrop (Microdrop Technologies GmbH, Muehlenweg 143, D-22844 Norderstedt, Germany). The conductive lines are for power supply to the sensors (2 conductive lines) as well as signal retrieval (8 conductive lines). On the opposite side of the strip, a number of lines as well as square islands are printed with a dimension of 1.6 mm wide by 2 mm long (the same spacing as the Hall sensors). The printed strips are cured for 30 minutes in a heated oven at 110 degrees Celsius.

PPy/Dex films are grown potentiostatically on the silver islands on the strip. A two electrode set-up is used. The electrochemical cell uses a 2 ml glass cuvette containing a working electrode (gold) and a platinum counter electrode. The coating process is controlled using the Gamry Potentiostat, FAS2/Femostat (Gamry Instruments) with Gamry framework software. The deposition solution (1 ml) contains 0.1 M pyrrole (Sigma) and 0.1 M dexamethasone disodium phosphate. In the potentiostatic mode, a constant potential of 1.8 V relative to the counter electrode is used. The amount of material deposited on the electrode surface is controlled by time via the total charge passed during deposition, 25 mC/cm2 charge density.

After depositing the EAP/drug layer, four Micro Hall sensors from Cryomagnetics, Oak Ridge, Tenn. (http://www.cryomagnetics.com/hall-effect-sensor.php), type HSU-1 are glued on the opposite site of the strip with a longitudinal distance of 2 mm between the sensors. A connection is made to the printed lines using conductive Silver Conductive Epoxy type 8330 (MG chemicals).

The strip is glued on the end to a balloon system with the Hall sensors positioned between the balloon and the strip, having the EAP layer facing outward. The remainder of the strip with printed wires is mounted on the catheter using a heat shrink tube (Advanced Polymers, 29 Northwestern Drive Salem, N.H. 03079-2838).

During operation, the Dexamethasone can be released from the EAP containers using a cyclic voltage, using a 100 mV/s between −0.8 and 1.4 Volt. Each individual island can be addressed individually upon analysis of the Hall sensor signal.

### EXAMPLE 2

A device as illustrated in FIGS. 7-10 can be made by first making a polyimide inner tube with four copper conducting wires inserted in the wall. Micro Hall sensors can be obtained from Cryomagnetics Oak Ridge, Tenn. (http://www.cryomagnetics.com/hall-effect-sensor.php). The type HSU-1 comes without packaging with a sensing area of 100 micrometers squared. The surrounding ceramic area can be further reduced in size by laser ablating this material away using a 193 nm excimer laser to a final size of 200 by 200 micrometers. The wires of the Hall sensors are soldered to the wires of the inner tube after removing 10 mm of the distal end of the polymer wall of the inner tube using the same excimer laser. Two square cavities are ablated out of the inner tube 10 mm and 20 mm proximal to the distal end with a depth of 0.04 inches to fit both sensors. Both holes are aligned axially. The sensors and wires are folded backwards over the polymer inner tube whereby the sensors are placed backside in the ablated cavities. A heat shrink tube (Advanced Polymers, http://www.advpoly.com/Products/ShrinkTubing/Catalog/ItemDetails.aspx?ItemNumber=029080CHGS&Units=inch, part no_{—}029080CHGS, expanded ID=0.029″) is shrunk (85 degrees C., hot air gun) over the distal end to seal the sensors and wires.

The inner tube with the sensors is fed through a Pebax 72D outer tube (ID 0.03″, OD 0.035″), leaving a section of 20 mm of the inner tube sticking out beyond the distal end of the outer tube. A non-compliant Pebax 72D balloon is attached by laser bonding to the Pebax outer tube and bonded with cyano acrylate to the polyimide inner tube. The catheter is finished with a hub to the proximal part after which the balloon is inflated at 1 atm. to be able to apply the drug coating. At the tangential place where the two sensors are aligned, the balloon is pat printed with a 50/50 mixture of paclitaxel and Iopromid over an axial section ranging the inner section between the two sensors. Finally, the balloon is folded and is ready for use.

During use, the patient is infused intravenously with a saline solution containing USPIO super-paramagnetic particles (Sinerem (Guerbet, Roissy, France)) at 2.6 mg/kg. Accumulation of these magnetic particles occurs in macrophages and inflamed plaques. See Trivedi, R A, “Identifying Inflamed Carotid Plaques Using In Vivo USPIO-Enhanced MR Imaging to Label Plaque Macrophages,” Arterioscler. Thromb. Vasc. Biol. 2006; 26:1601-1606. The patient is scanned by MRI to locate the vulnerable plaques, and a roadmap is produced to be able to place the balloon catheter under fluoroscopy near the detected sites. Axial movement and rotation allows the physician to superpose the coated section of the balloon against the vulnerable plaque after which the balloon is inflated at low pressure (2 atm.) to transfer the drug to the desired target site.

### EXAMPLE 3

A device as illustrated in FIGS. 14-15 is constructed by first making a polyimide inner tube with four conducting copper wires inserted in the wall as described in Example 2. Micro Hall sensors are attached to the inner tube in the same manner as in Example 2.

A tri-wing shaped soft silicon rubber piece (http://www.appliedsilicone.com/products-index.html, component part 40088) is cast and attached to an outer tube by using Loctite® 4981™ Super Bonder® Medical Device Adhesive. The rubber tri-shape has tipped wings such that upon retrieval in the delivery catheter they all will fold in the same direction. The three wings will make three channels (spaces between the wings), and one of them is closed by a silicon rubber membrane in place. In the valley of the closed chamber, one or more holes are punctured for the drug delivery ports.

The inner tube is fed through the outer tube and silicon wing shape and aligned such that the Hall sensors are located underneath the closed chamber after which the distal end of the inner tube is glued to the distal end of the outer part (the distal end of the expandable member). A soft rubber tip is glued to this assembly to finish off the product on the distal end. The space between outer tube and inner tube can now be used as a delivery lumen to inject a fluid (containing a drug) which then can emerge in the closed chamber through the drug delivery ports. In use, the closed chamber can be filled with a fluid drug content, while the other two chambers allow a continuous blood flow downstream to the distal part of the vessel.

Disclosed embodiments have been described with reference to several exemplary embodiments. There are many modifications of the disclosed embodiments which will be apparent to those of skill in the art. It is understood that these modifications are within the teaching of the present disclosure which is to be limited only by the claims.

What is claimed is:

1. A therapeutic agent delivery device comprising:
an elongate member having a distal end;
an expandable member disposed on the distal end of the elongate member; and
a drug delivery matrix disposed on at least a portion of the expandable member, the drug delivery matrix comprising:
one or more drug delivery areas, each drug delivery area comprising an electroactive polymer;
one or more sensors adapted to detect a condition of a target location on a vessel wall; and
one or more conductive elements for transmitting at least one signal from the one or more sensors and for transmitting at least one signal to the electroactive polymer of the one or more drug delivery areas;
wherein when a first sensor of the one or more sensors detects the condition of the target location, at least one signal is transmitted from the first sensor, and, based on the detection of the condition of the target location, at least one signal is transmitted to a first drug delivery area of the one or more drug delivery areas, thereby causing the therapeutic agent to be delivered from the electroactive polymer of the first drug delivery area to the target location.

2. The therapeutic agent delivery device according to claim 1, wherein the first drug delivery area is adapted to communicate with a second drug delivery area of the one or more drug delivery areas; and
wherein when the signal is transmitted to the first drug delivery area, the first drug delivery area communicates the signal to the second drug delivery area, thereby causing the therapeutic agent to be delivered from an electroactive polymer of the second drug delivery area to the target location.

3. The therapeutic agent delivery device according to claim 1, wherein the one or more sensors comprise at least one of a mechanical, environmental and biochemical sensor.

4. The therapeutic agent delivery device according to claim 1, wherein the signals are electrical signals.

5. The therapeutic agent delivery device according to claim 1, wherein the one or more conductive elements comprise at least one of metal and polymer wiring.

6. A method of delivering a therapeutic agent to a target location, the method comprising:
(a) using a therapeutic agent delivery device comprising:
(i) an elongate member having a distal end;
(ii) an expandable member disposed on the distal end of the elongate member; and
(iii) a drug delivery matrix disposed on at least a portion of the expandable member, the drug delivery matrix comprising:
(1) one or more drug delivery areas, each drug delivery area comprising an electroactive polymer;
(2) one or more sensors adapted to detect a condition of the target location on a vessel wall; and
(3) one or more conductive elements for transmitting at least one signal from the sensors and for transmitting at least one signal to the electroactive polymer of the one or more drug delivery areas;
(b) positioning the device in the vessel;
(c) detecting the condition of the target location; and
(d) transmitting at least one signal from a first sensor of the one or more sensors that detected the condition and, based on the detection of the condition at the target location, transmitting at least one signal to a first drug delivery area of the one or more drug delivery areas, thereby causing the therapeutic agent to be delivered from the electroactive polymer of the first drug delivery area to the target location.

7. The method of delivering a therapeutic agent according to claim 6, further comprising:
after the signal is transmitted to the first drug delivery area, communicating the signal from the first drug delivery area to a second drug delivery area, thereby causing the therapeutic agent to be delivered from an electroactive polymer of the second drug delivery area to the target location.

8. A method of delivering a therapeutic agent to a target location, the method comprising:
(a) determining one or more target drug delivery areas on a vessel wall;
(b) using a therapeutic agent delivery device comprising:
(i) an elongate member having a distal end;
(ii) an expandable member disposed on the distal end of the elongate member; and
(iii) a drug delivery matrix disposed on at least a portion of the expandable member, the drug delivery matrix comprising:
(1) one or more drug delivery areas, each drug delivery area comprising an electroactive polymer; and
(2) one or more conductive elements for transmitting at least one signal to the electroactive polymer of the one or more drug delivery areas;
(c) positioning the therapeutic agent delivery device in the vessel;
(d) transmitting at least one signal to one or more drug delivery areas, thereby causing the therapeutic agent to be delivered from the electroactive polymer layer of the one or more drug delivery areas to the target location.

9. The method of delivering a therapeutic agent according to claim 8, wherein the step of determining one or more target drug delivery areas on a vessel wall comprises pre-scanning the vessel wall using a scanning device.

10. The method of delivering a therapeutic agent according to claim 9, further comprising flushing the vessel with a detectable agent before pre-scanning the vessel wall.

11. The method of delivering a therapeutic agent according to claim 9, wherein the pre-scan is an X-Ray, CT, MRI or OCT scan.

12. The method of delivering a therapeutic agent according to claim 8, wherein the step of transmitting at least one signal occurs by automatically activating the drug delivery matrix.

13. The method of delivering a therapeutic agent according to claim 8, wherein the step of transmitting at least one signal occurs by manually activating the drug delivery matrix.

14. The method of delivering a therapeutic agent according to claim 11, wherein the detectable agent is a super-paramagnetic iron particle.

15. A therapeutic agent delivery device comprising:
an elongate member having a distal end;
an expandable member disposed on the distal end of the elongate member, the expandable member comprising a plurality of adjacent radially-expanding flexible walls that extend longitudinally in an axial direction along the length of the expandable member, the flexible walls forming a plurality of channels; and
a delivery lumen for delivering therapeutic agent to one or more of the plurality of channels;
wherein the therapeutic agent is delivered from the delivery lumen to one or more of the plurality of channels to a target location.

16. The therapeutic agent delivery device according to claim 15, wherein the expandable member has a retracted position in an outer catheter and an expanded position in a vessel; and
wherein the plurality of adjacent radially-expanding flexible walls are tapered at a proximal end of the expandable member to facilitate retraction of the expandable member from the expanded position in the vessel to the retracted position in the outer catheter.

17. The therapeutic agent delivery device according to claim 15, further comprising a plurality of sensors for locating the target location on a vessel wall.

18. A method of delivering a therapeutic agent to a target location, the method comprising:
(a) using a therapeutic agent delivery device comprising:
(i) an elongate member having a distal end;
(ii) an expandable member disposed on the distal end of the elongate member, the expandable member comprising a plurality of adjacent radially-expanding flexible walls that extend longitudinally in an axial direction along the length of the expandable member, the flexible walls forming a plurality of channels; and
(iii) a delivery lumen for delivering therapeutic agent; and
(b) delivering the therapeutic agent from the delivery lumen to a first channel of the plurality of channels to a target location.

19. The method of delivering a therapeutic agent according to claim 18, further comprising sensing the location of a target location on a vessel wall;
wherein the first channel of the plurality of channels is oriented adjacent the target location.

20. A method of determining the location of a lesion on a vessel wall, the method comprising:
(a) delivering a detectable agent to the vessel;
(b) using a lesion detection device comprising:
(i) an elongate member having a distal end; and
(ii) a plurality of sensors disposed on the distal end of the elongate member, the plurality of sensors adapted to sense the detectable agent;
(c) positioning the lesion detection device in the vessel; and
(d) determining a location of the lesion on the vessel wall based on signals received by the plurality of sensors from the detectable agent.
